(19) United States
(12) Patent Application Publication
(10) Pub. No.: US 2026/0097109 A1
(43) Pub. Date: Apr. 9, 2026

(54) HPV Virus-Like Particle Bioconjugates

(71) Applicant: Inventprise, Inc., Redmond, WA (US)

(72) Inventor: Subhash V. Kapre, Redmond, WA (US)

(73) Assignee: Inventprise, Inc., Redmond, WA (US)

(21) Appl. No.: 19/352,201

(22) Filed: Oct. 7, 2025

Related U.S. Application Data
(60) Provisional application No. 63/704,155, filed on Oct. 7, 2024.

(51) Int. Cl. A61K 39/12; A61K 39/00

(52) U.S. Cl. CPC A61K 39/12; A61K 2039/5258; A61K 2039/6037; A61K 2039/6075

(57) ABSTRACT

This invention is directed to immunogenic composition, vaccines and conjugates comprising virus-lie particles (VLP) compositions conjugated to one or more L2 proteins of HPV. The invention further includes methods for the manufacture of immunogenic compositions and vaccines and methods for the treatment and/or prevention of HPV infection.

### REFERENCE TO RELATED APPLICATIONS

This application claims priority to U.S. Provisional Application No. 63/704,155, filed Oct. 7, 2024, the entirety of which is incorporated by reference.

### BACKGROUND OF THE INVENTION

### 1. Field of the Invention

This invention is directed to immunogenic composition and vaccines composed of conjugates of virus-like particles (VLP) and various peptides or proteins, and methods directed to the manufacture of such immunogenic compositions and vaccines, and the treatment and/or prevention of infection by multiple types of Human Papillomavirus.

### 2. Description of the Background

Human Papillomavirus (HPV) is a group of more than 200 related which are designated by a number for each virus type. As a double stranded DNA virus, HPV's circular DNA genome is composed of two major oncogenes, E6 and E7, and two major structural protein genes, L1 and L2. Most conventional vaccines are developed based on these components. The L1 protein of HPV expressed recombinantly in vitro self-assembles into virus-like particles (VLPs). VLPs have HPV type-specific conformational neutralizing epitopes and are used for the development of VLP-based vaccine products. Typically, VLPs are recombinantly expressed in yeast, bacterial, or insect cell expression systems.

HPV targets the basal cells of squamous epithelia for infection. Some HPV types, such as HPV5, may establish infections that persist for the lifetime of the individual without ever manifesting any clinical symptoms. HPV types 1 and 2 can cause common warts in some infected individuals. HPV types 6 and 11 can cause genital warts and laryngeal papillomatosis.

Many HPV types are carcinogenic. About twelve HPV types (including types 16, 18, 31, and 45) are referred to a high-risk types because persistent infection has been linked to cancer of the oropharynx, larynx, vulva, vagina, cervix, penis, and anus. These cancers all involve sexually transmitted infection of HPV to the stratified epithelial tissue. HPV type 16 is the strain most likely to cause cancer and is present in about 47% of all cervical cancers, and in many vaginal and vulvar cancers, penile cancers, anal cancers, and cancers of the head and neck.

Human papillomavirus (HPV) vaccines provide acquired immunity against infection by certain types of human papillomavirus (HPV). The first HPV vaccine became available in 2006. Currently there are six licensed HPV vaccines: three bivalent (protect against two types of HPV), two quadrivalent (against four types), and one nonavalent vaccine (against nine types). All have excellent safety profiles and are highly efficacious, or have met immunobridging standards, meaning that the vaccine meets regulatory and scientific standards to infer vaccine effectiveness through comparison of immune response marker(s) elicited by a vaccine under different sets of conditions. All of these vaccines protect against HPV types 16 and 18, which are together responsible for approximately 70% of cervical cancer cases globally. The quadrivalent vaccines provide additional protection against HPV types 6 and 11. The nonavalent vaccine provides additional protection against HPV types 31, 33, 45, 52 and 58. HPV vaccines prevent approximately 70% of cervical cancer, approximately 80% of anal cancer, approximately 60% of vaginal cancer, approximately 40% of vulvar cancer, protect against penile cancer, and show more than 90% effectiveness in preventing HPV-positive oropharyngeal cancers. They additionally prevent genital warts (also known as anogenital warts), with the quadrivalent and nonavalent vaccines providing virtually complete protection. The vaccines require two or three doses depending on a person's age and immune status. The vaccines provide protection for at least five to ten years.

The primary target group in most of the countries recommending HPV vaccination is young adolescent girls, aged 9-14. The vaccines are particularly cost-effective in resource-constrained settings, especially within the GAVI vaccine alliance countries. The vaccination schedule depends on the age of the vaccine recipient. As of 2022, 125 countries include HPV vaccine in their routine vaccinations for girls, and 47 countries recommend them for boys, as well. Vaccinating a large portion of the population may also benefit the unvaccinated by way of herd immunity.[

The HPV vaccine is on the World Health Organization's List of Essential Medicines. The World Health Organization (WHO) recommends HPV vaccines as part of routine vaccinations in all countries, along with other prevention measures. The WHO's priority purpose of HPV immunization is the prevention of cervical cancer, which accounts for 82% of all HPV-related cancers. In 2020, 88% of cervical cancers occurred in low- and middle-income countries and 2% in high-income countries. The WHO-recommended primary target population for HPV vaccination is girls aged 9-14 years before they become sexually active. Females aged greater than 15 years of age, boys, and older males are secondary target populations. Cervical cancer screening is still required following vaccination.

Two HPV vaccines are currently on the market, Gardasil (Merck and Co. Inc.) and Cervarix (GSK). The composition and dose of the Gardasil vaccine comprises HPV VLP L1 protein containing 6, 11 ,16 ,18 ,1, 33, 45, 52, 58, a total of 9-serotypes and an aluminum adjuvant. The VLPs are present in an amount of 20-40μg each per dose. The vaccine is administered as a 3-dose regimen according to a 0, 2, and 6-month schedule. The Cervarix vaccine comprises HPV VLPs 16 and 18 L1 proteins, and an adjuvant containing aluminum hydroxide and MPLA (3D-MPL). The VLPs are present at 20μg each per dose. This vaccine is also administered as a 3-dose regimen according to a 0, 2, and 6-month schedule.

The cost of vaccine and the limited nine valency remain as blocks in immunizing greater population in the developing world. Thus, a lower cost and more widely protective vaccine is in great need throughout the world.

### SUMMARY OF THE INVENTION

The present invention overcomes the problems and disadvantages associated with current strategies and designs and provides new immunogenic compositions, method of manufacturing immunogenic compositions, and methods of treated and preventing infections with the immunogenic compositions.

One embodiment of the invention is directed to immunogenic compositions composed of virus-like particles (VLPs) obtained or derived from L1 proteins of Human papilloma virus (HPV), conjugated with multiple HPV L2 proteins, also referred to herein as bioconjugation. Preferably the HPV L2 protein is derived from one or more HPV types and/or subtypes. Preferably the composition contains spacer arms, which may contain a hetero-or homo-bifunctional or multifunctional spacer arms, and carrier protein, which may contain tetanus toxoid, diphtheria toxoid, CRM197, tetanus toxoid fragments (TTHc), N. meningitidis protein PorB, RSV virus proteins, B. Pertussis proteins, Pertussis toxoid (PT), adenylate cyclase toxin (ACT), 69 KDa protein, Human Papilloma viral protein antigens, Human Papilloma virus VLP forms, Hepatitis B virus core antigen, Hepatitis B virus VLP forms, derivatives of HBsAg, and/or combinations thereof. Preferably the immunogenic composition comprises an adjuvant, and preferably the adjuvant comprises aluminum salt, calcium phosphate, a liposome of monophosphoryl lipid A (MPLA), saponin QS-21, TLR ligands, and/or a potent TLR4/7/8/9 agonists. Preferred aluminum salts include one or more of aluminum phosphate, aluminum sulfate and/or aluminum hydroxide. Preferably the immunogenic composition, when administered to a patient, boosts the efficacy of a conventional vaccine.

Another and related embodiment of the invention comprises the process of manufacturing the immunogenic composition of the disclose. Preferably the immunogenic composition is manufactures by the conjugation of VLPs of HPV L1 protein with HPV L2 protein. Preferably the conjugation is mediated with an enzyme, such as, for example, any of the sortase enzymes. Also preferably the L1 and/or L2 proteins are modified for click chemistry. Modifications can include coupling chemical motifs to the proteins for ease of conjugation.

Another and related embodiment of the invention is the treatment of an HPV infection by administration of the immunogenic composition disclosed herein. Preferably the immunogenic compositions and stable and provide protection against infections at lower doses or less frequently than are available using conventional immunogenic compositions. Preferably, a single dose provides sufficient and effective protection against HPV infection attributed to multiple types of HPV.

Other embodiments and advantages of the invention are set forth in part in the description, which follows, and in part, may be obvious from this description, or may be learned from the practice of the invention.

### DESCRIPTION OF THE INVENTION

HPV targets the basal cells of squamous epithelia for infection. There are two proteins that are important in the treatment of HPV infection. One is a protein that is mostly common to various subtypes and is called L2, the other is termed L1 which has varied structures. These proteins are denoted as L18 and L16, and form VLPs which are utilized as antigens in the manufacture of a vaccine. Some HPV types, such as HPV5, may establish infections that persist for the lifetime of the individual without ever manifesting any clinical symptoms. HPV vaccines are available that provide acquired immunity against infection by certain types of human papillomavirus (HPV). Currently available vaccines have excellent safety profiles and are highly efficacious. These vaccines require two or three doses depending on a person's age and immune status, provide protection for at least five to ten years, and are effective against up to nine types of HPV.

An immunogenic composition has been surprisingly discovered that provides HPV immunity equivalent to conventional HPV vaccines to against a large number of HPV types and subtypes. The composition is more broadly effective against HPV infection than conventional vaccines making the vaccine of this disclosure more practical and, with the expected reduced costs, more widely available. AS the components of the immunogenic composition are nearly identical to the components of conventional vaccines, they are also safe and effective.

Immunogenic compositions of this disclosure comprise conjugating HPV L1 VLPs with one or more, preferably multiple types of the L2 protein of HPV. This generates a broader cross-protective immune response against different HPV serotypes. The L1 protein self-assembles into VLPs and mimics the native viral capsid structure, while the L2 protein contains conserved epitopes across different HPV types, making this an ideal target for creating a pan-HPV vaccine. A conjugate of VLPs composed of the L1 protein can be conjugated with an enzyme to one or multiple types of the L2 protein to form an effective immunogenic composition and vaccine. Conjugation is preferably performed with a sortase enzyme, preferably sortase A, transglutaminase-mediated conjugation, or tyrosinase-mediated conjugation. The strategies to conjugate L1 VLPs with the L2 protein using enzymatic or chemical approaches include mediated conjugation which may include the advantages of Click Chemistry.

Immunogenic compositions of this constructions are useful in the treatment and prevention of many different types of HPV infections, many more than are currently available with commercially available vaccines. As the bioconjugation of VLPs can be performed with multiple types and/or subtypes of HPV molecules, each type and subtype can provide protection against infection attributed to the corresponding HPV types and subtypes. Enzyme and the reaction conditions are chosen based on the type of bond desired (e.g., amide bond, disulfide bond) and the stability required. The structural integrity of the resulting VLP is such that conjugation does not interfere with self-assembly or biological function, and specific and stable conjugation of a VLP to another protein is achieved, enabling the formation of novel bioconjugates that can be utilized as vaccines.

Preferably, the conjugation takes advantage of click chemistry, an approach to chemical synthesis that emphasizes efficiency, simplicity, selectivity, and modularity in chemical processes used to join molecular building blocks. By way of a non-limiting example, VLP can genetically engineered with an LPXTG motif after which Sortase A attaches an azide and/or alkyne-containing peptide to the VLP. The complementary alkyne or azide group is then attached to the target protein and CuAAC to covalently bond the VLP and protein. This method is highly versatile, allowing precise control over the conjugation process and enabling the attachment of a wide range of molecules. This method ensures that the reactive groups (e.g., lysine, glutamine, tyrosine, or cysteine) are surface-exposed on both the VLP and the protein to be conjugated.

The disclosure is directed to pharmaceutical compositions for the treatment and prevention of Human papilloma virus (HPV) infections such as, for example infections that cause various forms of cancer, comprising of virus-like particles (VLP) preferably derived from L1 HPV clones, conjugated with one or more types and/or subtypes of L2 proteins. The conjugate combination comprises combinations of types and subtypes of the L2 protein prevalent in regions where infections of those types exist. Preferably the immunogenic composition comprises one or more HPV serotypes, two one or more HPV serotypes, three or more HPV serotypes, four or more HPV serotypes, five or more HPV serotypes, six or more HPV serotypes, seven or more HPV serotypes, eight or more HPV serotypes, nine or more HPV serotypes, ten or more HPV serotypes, fifteen or more HPV serotypes, twenty or more HPV serotypes, twenty five or more HPV serotypes, thirty or more HPV serotypes, or even more different serotypes. Preferably, compositions include carrier proteins, spacers and/or adjuvants. Preferred carrier proteins include tetanus toxoid, diphtheria toxoid, CRM197, tetanus toxoid fragments (TTHc), N. meningitidis protein PorB, RSV virus proteins, B. Pertussis proteins, Pertussis toxoid (PT), adenylate cyclase toxin (ACT), 69 KDa protein, Human Papilloma viral protein antigens, Human Papilloma virus VLP forms, Hepatitis B virus core antigen, Hepatitis B virus VLP forms, derivatives of HBsAg, and/or combinations thereof. Preferred spacers comprises a hetero- or homo-bifunctional or multifunctional spacer arm. Preferably the immunogenic composition comprises an adjuvant, and preferably the adjuvant comprises aluminum salt, calcium phosphate, a liposome of monophosphoryl lipid A (MPLA), saponin QS-21, TLR ligands, and/or a potent TLR4/7/8/9 agonists. Preferred aluminum salts include one or more of aluminum phosphate, aluminum sulfate and/or aluminum hydroxide. Preferably the immunogenic composition, when administered to a patient, boosts the efficacy of a conventional vaccine. Preferably an immunogenic composition of this disclosure comprises VLP of HPV L1 protein conjugated to HPV L1 protein derived from ten or more serotypes of HPV, which also preferably contains carrier protein such as CRM197. Pharmaceutical compositions may further include a pharmaceutically acceptable carrier such as water, oil, dyes, colorants, flavoring agents, stabilizers, anti-foaming agents, alcohols, and other acceptable materials.

In addition, the present disclosure includes formulation for at least 9-, 10-, 15-, 20, 25- or higher valent VLP conjugate vaccine which reduces the necessary dose (8-10μg/dose of individual VLPs compared with 20-40μg dose used in Gardasil, Merck or Cerverix, GSK) adsorbed in aluminum phosphate or aluminum hydroxide or other suitable TLR7/8/9 or TLR 4 adjuvants.

Another and related embodiment comprises the manufacture of immunogenic compositions of the disclosure. Manufacture involves a recombinant L2 protein with motif such as a LPXTG motif, which when mixed with the VLPs of L1 protein and an enzyme Sortase A to create a bioconjugate. Bioconjugate are important to prevent any damage to the VLPs and prove to be a better vaccine than unconjugated HPV vaccines and enhances the number of serotypes antigens that are available as a platform technology.

Another and related embodiment comprises administration of composition and vaccines of the disclosure to patients. Administration may be oral, parenteral, IV, IM, or any other route that provides an effective immunogenic response to an HPV infection. Preferably, the dosage to an individual is from 8-10μg composition dry weight. Also preferably, a single dose provides sufficient and effective treatment and/or protection against the desired form of HPV infection.

The following examples illustrate embodiments of the invention but should not be viewed as limiting the scope of the invention.

### EXAMPLES

### Example 1 Sortase Plus Click Chemistry Dual Approach

As an initial step, sortase (a group of enzymes that modify surface proteins of Gram-positive bacteria by attaching them to the cell wall) is combined with click chemistry to enhance specificity. First, Sortase A is used to site-specifically attach a functional group (e.g., an alkyne or azide) to either the VLP or the target protein. Then, click chemistry (e.g., copper-catalyzed azide-alkyne cycloaddition, CuAAC) is used to covalently bond the VLP and protein. In one example, VLP is genetically engineered with an LPXTG motif after which Sortase A attaches an azide and/or alkyne-containing peptide to the VLP. The complementary alkyne or azide group is then attached to the target protein and CuAAC to covalently bond the VLP and protein. This method is highly versatile, allowing precise control over the conjugation process and enabling the attachment of a wide range of molecules. This method ensure that the reactive groups (e.g., lysine, glutamine, tyrosine, or cysteine) are surface-exposed on both the VLP and the protein to be conjugated.

The enzyme and the reaction conditions are chosen based on the type of bond desired (e.g., amide bond, disulfide bond) and the stability required. The structural integrity of the resulting VLP is such that conjugation does not interfere with self-assembly or biological function, and specific and stable conjugation of a VLP to another protein is achieved, enabling the formation of novel bioconjugates that can be utilized as vaccines.

### Example 2-Examples of Conjugating HPV L1 VLPS With L2 Protein of HPV

Conjugating HPV L1 VLPs (Virus-Like Particles) with the L2 protein of HPV generates a broader cross-protective immune response against different HPV serotypes. The L1 protein self-assembles into VLPs and mimics the native viral capsid structure, while the L2 protein contains conserved epitopes across different HPV types, making this an ideal target for creating a pan-HPV vaccine. The strategies to conjugate L1 VLPs with the L2 protein using enzymatic or chemical approaches include:

### 1. Sortase-mediated Conjugation

Sortase A is used to site-specifically conjugate the L2 protein to HPV L1 VLPs by introducing appropriate peptide motifs. L1 protein is engineered to contain a C-terminal LPXTG motif, which is recognized by Sortase A. The VLPs will form as usual with this additional tag. The L1 VLPs with the LPXTG tag is purified expressed with an N-terminal glycine (Gly) tag, which serves as the target for Sortase A. Sortase A conjugation involves mixing the purified L1 VLPs and L2 protein in the presence of Sortase A. Sortase A will cleave between the threonine and glycine residues of the LPXTG motif on L1 and form a covalent bond between the L2 protein's N-terminal glycine and the threonine of L1 and the conjugate product purified.

This method results in highly specific L1-L2 conjugates, wherein L2 proteins are covalently attached to the surface of the VLP, which allows the L2 protein's conserved epitopes to be displayed in a multivalent format.

Sortase-mediated ligation has been used to link peptides and proteins to the surface of VLPs for vaccine development, such as with norovirus VLPs, and the same principle can be applied to HPV L1-L2 conjugation.

### 2. Chemical Crosslinking via Nhs-esters and Maleimides

A common chemical crosslinking approach can be used when site-specific conjugation is not strictly necessary, but a covalent bond is preferred. To functionalize L1 VLPs, a heterobifunctional crosslinker, for example, SMCC (Succinimidyl 4-(N-maleimidomethyl) cyclohexane-1-carboxylate), is used to functionalize the L1 VLP. SMCC contains an NHS-ester group that reacts with lysine residues on the L1 VLPs and a maleimide group for conjugation with cysteines on the L2 protein. The L2 protein is engineered with a C-terminal cysteine or ensure that an exposed cysteine residue is present and purified. The conjugation reaction is performed on the SMCC-modified L1 VLP mixed with the cysteine-containing L2 protein. The maleimide group on SMCC reacts with the thiol group on L2's cysteine, forming a stable thioether bond and the L1-L2 conjugated VLP is purified. This method is robust for generating covalent L1-L2 conjugates and is widely used in general vaccine development. The method can also be scaled up and optimized for different stoichiometries of L2 attachment to the VLP surface.

As an example, NHS-ester and maleimide crosslinking methods are commonly used to conjugate proteins in vaccine development and drug delivery systems.

### 3. Transglutaminase-mediated Conjugation

Transglutaminase is used to catalyze a covalent bond between lysine and glutamine residues on the L1 VLP and L2 protein. The L1 VLP is prepared by lysine residues on the surface of the L1 VLP which are identified and/or introduced accessible lysines through mutagenesis. The L2 protein is engineered to ensure that L2 has surface-exposed glutamine residues. Transglutaminase reaction is performed by incubating the L1 VLPs and the L2 protein with transglutaminase. The enzyme catalyzes a covalent bond between the lysine residue on the L1 VLP and the glutamine residue on the L2 protein and the conjugated L1-L2 VLP purified.

This approach provides a stable covalent attachment, which is important for producing highly stable L1-L2 conjugates for vaccines.

Transglutaminase has been used for crosslinking proteins and is compatible with creating stable multimeric constructs like VLP-protein conjugates.

### 4. Tyrosinase-mediated Conjugation

Tyrosinase catalyzes the formation of dityrosine bonds between tyrosine residues on the L1 VLP and L2 protein. The accessible of tyrosine residues are identified on the surface of the L1 VLP and L2 protein. The L1 VLPs and L2 protein are incubated with tyrosinase under mild oxidative conditions. Tyrosinase oxidizes the tyrosine residues, leading to the formation of covalent dityrosine bonds between the L1 and L2 proteins and the conjugated product is purified.

This method provides strong, covalent linkages that are ideal for generating stable vaccines and vaccine candidates.

Tyrosinase-based crosslinking has been applied to conjugate antigens to VLPs, making it applicable to L1-L2 conjugation for HPV vaccines.

For conjugating HPV L1 VLPs with the L2 protein, sortase-mediated conjugation provides site-specific attachment with minimal disruption to the VLP structure. Chemical crosslinking with NHS-ester/maleimide chemistry provides flexibility, but may be less specific. Transglutaminase and tyrosinase approaches offer enzymatic routes for stable, covalent attachment.

VLP has N-Terminal glycines that can be used to create the LPXTG on L2 protein and carry out the conjugation.

VLP (in this case, HPV L1 VLPs) has N-terminal glycines create an LPXTG motif on the L2 protein. Sortase A can then be used to conjugate the L2 protein to the VLPs. This approach takes advantage of the natural N-terminal glycine on the VLP, which Sortase A can use as the nucleophile to form a covalent bond with the L2 protein's engineered LPXTG motif.

### Example 2-Examples of Engineering the L2 Protein With Lpxtg Motif

Genetically engineer the L2 protein to include a C-terminal LPXTG motif, which is the recognition sequence for Sortase A. The sequence LPETG (which is commonly used in Sortase A-mediated conjugation) is added at the C-terminus of the L2 protein during expression. The L1 protein of the HPV VLP can have exposed N-terminal glycine residues, which can serve as the target for the Sortase A reaction for preparation of the L1 VLPs with N-terminal Glycines. These N-terminal glycines are accessible and not buried within the VLP structure. Preferably, a purification method can be used that ensures N-terminal glycines are free for the conjugation reaction.

### 1. Sortase A-mediated Conjugation

The L1 VLPs and L2 protein are purified with the LPETG tag. The L1 VLPs and L2 protein are incubated with Sortase A enzyme. The reaction typically takes place at room temperature or slightly elevated temperatures (25-37° C.) in a suitable buffer (e.g., Tris-HCl, Ca²⁺-containing buffer, pH 7.5-8.0). Sortase A will cleave between the threonine (T) and glycine (G) in the LPXTG motif of L2. The resulting intermediate forms a covalent bond between the threonine of the L2 protein and the N-terminal glycine of the L1 VLP. After the reaction, purify the conjugated L1-L2 product to remove any unreacted L2 protein and Sortase A enzyme. The conjugation reaction is highly specific, attaching the L2 protein only to the N-terminal glycines on the VLP. Since only the N-terminus of the VLP is involved in the conjugation, the overall structure and integrity of the VLP remain intact with minimal or no disruption to VLP structure. Sortase A reactions are well-established for scaling up, and you can optimize the ratio of L2 to VLP for efficient conjugation. N-terminal glycines on the VLP are mostly accessible and Sortase A works well with short sequences of glycines, so the N-terminal glycine are preferably close to the surface of the VLP for efficient conjugation. The concentration of VLP, L2, and Sortase A is optimized for high yield and efficiency.

The conjugation is analyzed using techniques such as SDS-PAGE, Western blotting, or mass spectrometry to verify that the L2 protein has successfully been attached to the L1 VLP. This strategy allows for the generation of a site-specific, covalently conjugated L1-L2 particle, which can potentially enhance the immunogenicity of your HPV vaccine candidate by presenting conserved L2 epitopes in a multivalent format.

To insert the LPXTG motif into the L2 protein for Sortase A-mediated conjugation, the L2 protein is genetically modified to include this recognition sequence. Typically, the LPXTG motif is inserted at the C-terminus of the L2 protein because Sortase A cleaves between the threonine (T) and glycine (G) of this motif and facilitates conjugation to an N-terminal glycine on the partner protein (such as your VLP). To Insert LPXTG into L2 Protein, the gene sequence of the L2 protein is deytermined with the sequence of the L2 protein, and its DNA modified to add the LPXTG motif. The C-terminal end of the L2 gene is modified to add the sequence for the LPXTG motif (or more commonly, LPETG for Sortase A reactions) such as, for example:
Original L2 gene: 5′-ATG . . . [L2 coding sequence]. . . TAA (stop codon)-3′
Modified L2 gene: 5′-ATG . . . [L2 coding sequence]. . . TTG CCT GAA ACC GGA TAA (LPETG followed by stop codon)-3′
   LPXTG Codons include:
L: CTG
P: CCT
X (for E): GAA
T: ACC
G: GGA

Additional linkers (such as GGS for flexibility) can be added between the L2 protein and the LPXTG tag as desired.

Synthesis of the modified L2 gene with the LPXTG tag is commercial available from various commercial companies. The sequence containing the L2 protein is provided followed by the LPXTG motif. With the L2 gene in a plasmid, site-directed mutagenesis introduces the LPXTG sequence at the C-terminus. Commercially available kits are used for site-directed mutagenesis (such as QuikChange), which allow for precise modifications in a gene without re-cloning the entire construct. Example primers for site-directed mutagenesis (assuming addition at the C-terminus):
Forward primer: 5′- . . . [last L2 codons] TTG CCT GAA ACC GGA-3′
Reverse primer: 5′- . . . [reverse complement of last L2 codons] TCC GGT TTC AGG CAA-3′

### 2. Cloning of the Modified Gene Into an Expression Vector

The synthesized or mutated L2 gene to include the LPXTG tag is enclosed into an appropriate expression vector (e.g., pET, pcDNA, or any suitable vector for bacterial or mammalian expression). Preferably the vector has the correct regulatory elements (promoter, ribosome binding site, etc.) for high-level expression of the L2-LPXTG fusion protein in the chosen host system (e.g., E. coli, insect cells, or mammalian cells). The expression vector containing the modified L2 gene (with the LPXTG motif) is transformed into the host organism (e.g., E. coli). The expression conditions are modified for high yield of soluble L2 protein. In some cases, adding solubility tags like His-tag or GST can help with purification and solubility. The L2 protein is purified using standard protein purification techniques (e.g., affinity chromatography, ion exchange chromatography, or size exclusion chromatography). For a purification tag (e.g., His-tag), uses a corresponding affinity resin (e.g., Ni-NTA) to purify the protein.

### 3. Verification of Lpxtg Insertion

The L2 protein contains the LPXTG sequence is confirmed by sequencing the plasmid to ensure the insertion was successful. Verification of the presence of the LPXTG motif is performed by running mass spectrometry or Western blotting with specific antibodies against the tag (with an included tag).

### 4. Sortase a Conjugation

Once the L2 protein with the C-terminal LPXTG tag is expressed and purified, it is conjugated to the N-terminal glycine of the L1 VLP using Sortase A by mixing the L2-LPXTG protein and the L1 VLPs (with N-terminal glycines) in a suitable buffer (e.g., Tris-HCl, pH 7.5, with Ca²⁺, which is necessary for Sortase A activity). Sortase A is added to the reaction mixture. The enzyme will cleave between the threonine (T) and glycine (G) in the LPXTG motif on the L2 protein and form a covalent bond with the N-terminal glycine on the L1 VLP. The conjugated VLP-L2 product is purified, removing any excess L2 protein and Sortase A. Example Vector and Tags include pET or pGEX plasmids are commonly used for bacterial expression; Tags such as His-tag allow for easy purification or FLAG-tag for detection, which can be placed at either end of the L2 protein, ensuring the LPXTG motif is accessible at the C-terminus. Inserting the LPXTG motif at the C-terminus of the L2 protein is straightforward using gene synthesis or site-directed mutagenesis. Once the L2 protein is expressed and purified, Sortase A-mediated conjugation can be carried out with N-terminal glycine-containing VLPs for a highly specific, covalent linkage. This method ensures that the L2 protein is displayed on the VLP surface in a controlled and stable manner.

### 5. Sortase A Synthesis

To synthesize Sortase A, express and purify using recombinant DNA technology. Sortase A is a bacterial enzyme (originally from Staphylococcus aureus) that cleaves between the threonine (T) and glycine (G) in the LPXTG motif, catalyzing the formation of covalent bonds between proteins. Either clone the Sortase A gene from Staphylococcus aureus (gene ID: srtA) or purchase a synthetic gene or use a plasmid containing the srtA gene. The mature form of Sortase A, which is about 206 amino acids long (after cleavage of the signal peptide). The gene coding for this truncated version can be used for expression in bacteria. The Sortase A gene sequence, mature form, codon-optimized for E. coli, if desired) includes the region encoding amino acids 60-206 (mature active form of Sortase A). Vector Design involves cloning the srtA gene into a suitable expression vector such as pET28a or pET21b, designed for high-level expression in E. coli, preferably with an N-terminal His-tag (e.g., 6×His) for easy purification using nickel affinity chromatography. The His-tag won't interfere with the enzyme activity and can later be cleaved off if necessary. A construct involves vbnet with copy code as promoter—RBS—6×His-srtA—Stop Codon. The transform of an E. coli strain (commonly used strains include BL21(DE3) or Rosetta(DE3)) with the plasmid containing the srtA gene. Transformed cells are selected using antibiotics corresponding to the plasmid's resistance marker (e.g., kanamycin for pET28a, ampicillin for pET21b). A fresh culture of transformed E. coli into LB or terrific broth (TB) is inoculated containing the appropriate antibiotic, the culture grown at 37° C. to OD600˜0.6-0.8, protein expression is induced with 0.5-1 mM IPTG (isopropyl β-D-1-thiogalactopyranoside), the temperature shifted to 16°C.-18°C. for overnight expression to promote proper folding and solubility. After expression, cells are harvested by centrifugation (5,000g for 10 minutes at 4° C.). The cell pellet is resuspended in lysis buffer comprising 50 mM Tris-HCl, pH 7.5; 150 mM NaCl; 5 mM imidazole (for His-tag binding); 1 mM DTT (optional, to prevent oxidation); add protease inhibitors (such as PMSF or a protease inhibitor cocktail) to protect Sortase A from degradation. The cells are lysed using sonication or a high-pressure homogenizer.

### 6. Protein Purification (Affinity Chromatography)

The Sortase A enzyme is purified using the His-tag. The cell lysate is loaded onto a Ni-NTA column (nickel-nitrilotriacetic acid affinity column) pre-equilibrated with lysis buffer. The column is washed with wash buffer (to remove non-specifically bound proteins) containing: 50 mM Tris-HCl, pH 7.5; 150 mM NaCl; 20 mM imidazole. Sortase A is eluted using an elution buffer with a higher imidazole concentration: 50 mM Tris-HCl, pH 7.5; 150 mM NaCl; 250-300 mM imidazole. The eluted fractions is collected and analyzed them by SDS-PAGE to verify the purity of Sortase A. After elution, imidazole is removed and buffer conditions adjusted for activity assays or storage. A dialysis membrane (with cut-off˜10 kDa) or a size-exclusion column is used to exchange the buffer into the desired storage buffer: 50 mM Tris-HCl, pH 7.5; 150 mM NaCl; 5 mM CaCl₂ (essential for Sortase A activity). The purified Sortase A is concentrated using a centrifugal concentrator (e.g., Amicon Ultra) to the desired concentration. Removal of the His-tag includes a protease cleavage site (e.g., TEV protease site) between the His-tag and Sortase A in the plasmid design. After purification, the protein is treated with TEV protease to cleave off the His-tag, followed by a secondary purification step to separate the cleaved His-tag. The purified Sortase A enzyme is aliquoted and stored at −80° C. for long-term storage. If using the enzyme for experiments within a few days, storage is at 4° C. Additives like 10% glycerol can be included to improve storage stability. To cleave the His-tag from a protein, include a protease cleavage site between the His-tag and the protein of interest during the cloning process. A commonly used site is the TEV protease cleavage site (ENLYFQ|G), which is recognized and cleaved by TEV protease.

### 7. Ensure Presence of the His-tag and Cleavage Site

When designing the plasmid, a protease cleavage site is included between the His-tag and the protein. Popular proteases and their cleavage sites are:
TEV protease: Cleaves after the sequence ENLYFQ|G.
Thrombin: Cleaves after the sequence LVPR|GS.
Factor Xa: Cleaves after the sequence IEGR|.
Example sequence: His-tag+TEV site+Sortase A.
   With this structure, the expression construct will have the correct protease recognition site for efficient cleavage. Purification of protein containing the His-tag and cleavage site is performed using nickel affinity chromatography as previously described. The His-tag allows binding to the protein to a Ni-NTA column and purification.

### 8. Protease Cleavage Reaction

After purification, the cleavage reaction is performed, first, with dialyze or exchange the buffer of the eluted protein into a suitable buffer for protease cleavage. For example, with TEV protease, the buffer is: 50 mM Tris-HCl, pH 7.5; 150 mM NaCl; 0.5 mM EDTA; 1 mM DTT (optional, for TEV protease stability); plus protease in a 1:50 to 1:100 ratio (w/w) of protease to target protein. For example, with 1 mg of protein, about 10-20μg of TEV protease is included. The reaction is incubated at 4° C. or 25° C. (depending on the protease) for 2-16 hours. TEV protease is active at both 4° C. and room temperature, but a slower reaction at 4° C. may reduce the risk of protein degradation. The Cleaved Protein is purified so that after the cleavage reaction, there will be a mixture of cleaved target protein (without the His-tag); His-tag; and protease (if it has a His-tag). Purification of the cleaved protein involves loading the reaction mixture back onto a Ni-NTA column (nickel affinity column). The cleaved His-tag and His-tagged protease will bind to the column, while the target protein (without the His-tag) will flow through. The flow-through is collected, which contains the target protein with the His-tag removed. If the protease doesn't have a His-tag, add an additional purification step (e.g., size exclusion chromatography) to remove the protease. To check the cleavage efficiency and protein purity using SDS-PAGE or Western blot. There will be a shift in molecular weight corresponding to the removal of the His-tag (the difference will depend on the length of the His-tag and linker region).

Cleavage reaction conditions: protein (His-tagged with TEV site) at 1-5 mg/mL; TEV protease at 1:50 to 1:100 ratio (w/w) of protease to protein; cleavage buffer comprising 50 mM Tris-HCl, pH 7.5; 150 mM NaCl; 0.5 mM EDTA; 1 mM DTT (optional), all to incubate at 4° C. or room temperature for 2-16 hours. After Cleavage, the reaction is reloaded onto a Ni-NTA column and the flow-through containing the cleaved, His-tag-free protein collected. SDS-PAGE or Western blot is used to verify the removal of the His-tag. By following this method, obtain the target protein without the His-tag, ready for downstream applications.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. It is intended that the specification and examples be considered exemplary only with the true scope and spirit of the invention indicated by the following claims. Furthermore, the term “comprising of” includes the terms “consisting of” and “consisting essentially of. ”

1. An immunogenic composition comprising virus-like particles (VLPs) of HPV L1 protein conjugated to one or more types of HPV L2 protein.

2. The immunogenic composition of claim 1, wherein the one or more types of HPV L2 protein comprise five or more, ten or more, fifteen or more, or twenty or more different antigenic types of L2 protein.

3. The immunogenic composition of claim 1, further comprising a carrier protein.

4. The immunogenic composition of claim 3, wherein the carrier protein comprises tetanus toxoid, diphtheria toxoid, CRM197, tetanus toxoid fragments (TTHc), N. meningitidis protein PorB, RSV virus proteins, B. Pertussis proteins, Pertussis toxoid (PT), adenylate cyclase toxin (ACT), 69 KDa protein, Human Papilloma viral protein antigens, Human Papilloma virus VLP forms, Hepatitis B virus core antigen, Hepatitis B virus VLP forms, derivatives of HBsAg, and/or combinations thereof.

5. The immunogenic composition of claim 1, further comprising one or more hetero-or homo-bifunctional or multifunctional spacer arms.

6. The immunogenic composition of claim 1, further comprising an adjuvant.

7. The immunogenic composition of claim 6, wherein the adjuvant comprises aluminum salt, calcium phosphate, a liposome of monophosphoryl lipid A (MPLA), saponin QS-21, TLR ligands, and/or a potent TLR4/7/8/9 agonists.

8. The immunogenic composition of claim 7, wherein the aluminum salt is selected from the group consisting of aluminum phosphate, aluminum sulfate and/or aluminum hydroxide.

9. The immunogenic composition of claim 1, wherein the VLPs comprise L1 proteins of HPV serotypes 16 and/or 18.

10. The immunogenic composition of claim 1, wherein the one or more types of HPV L2 protein comprise serotypes 6, 11, 16, 18, 31, 33, 45, 52, and/or 58.

11. A method of manufacture of the composition of claim 1 comprising:
providing VLPs of HPV L1 protein;
providing HPV L2 proteins;
conjugating the VLPs with the HPV L2 proteins, or antigenic portions thereof, with an enzyme forming the composition; and
isolating the composition.

12. The method of claim 12, further comprising adding an adjuvant.

13. The method of claim 12, further comprising adding a carrier protein.

14. The method of claim 12, further comprising adding a pharmaceutically acceptable carrier.

15. A method of treating or preventing an HPV infection comprising:
administering the composition of claim 1 to a patient in need thereof.
